(19) United States
(12) Patent Application Publication
(10) Pub. No.: US 2007/0219527 A1
(43) Pub. Date: Sep. 20, 2007

(54) Catheter including a catheter valve, method of at least partially coating a catheter valve surface, and an apparatus for at least partially opening a catheter valve

(76) Inventor: William R. Barron, Riverton, UT (US)

Correspondence Address: STOEL RIVES LLP - SLC, 201 SOUTH MAIN STREET, ONE UTAH CENTER, SALT LAKE CITY, UT, 84111, US

(21) Appl. No.: 11/647,521

(22) Filed: Dec. 28, 2006

Related U.S. Application Data
(60) Provisional application No. 60/755,392, filed on Dec. 30, 2005.

(51) Int. Cl. A61M 25/00

(52) U.S. Cl. 604/523

(57) ABSTRACT

A catheter including a body defining a lumen and a valve including a perforation which forms a plurality of catheter valve surfaces. At least one coating may be substantially permanently adhered to at least a portion of at least one of the plurality of catheter surfaces. Also, a method of at least partially coating a catheter valve surface is disclosed. Further, the catheter valve may comprise a perforation which forms a plurality of catheter surfaces. At least one coating may be substantially permanently adhered to at least a portion of at least one of the plurality of catheter valve surfaces. An apparatus for at least partially opening a catheter valve is disclosed, including a tubular member including a bent region and an opening formed generally proximate the bent region structured to cause a catheter valve of a catheter to at least partially open.

This application claims priority to the previously filed provisional application Ser. No. 60/755,392, filed Dec. 30, 2005. This application is incorporated herein by reference.

### BACKGROUND

Catheters are commonly used to access a vascular system of a patient from outside the body of the patient. The cardiovascular access afforded by such catheters permits, for example, the monitoring of blood pressure, the aspiration of blood, and the infusion of medicaments and nutrients at various locations within the cardiovascular system. For example, catheters may provide access to the central regions of the cardiovascular system in the vicinity of the high volume blood flow passageways immediately interconnected with the heart (e.g., a vena cava).

Conventional vascular access catheters have been provided with closed distal ends and selectively operable valve structure(s) formed through the catheter body. These valve structures may be opened during therapeutic fluid infusion or aspiration, but may remain closed when the catheters are not in use. One embodiment of a valve structure developed for this purpose takes the form of one or more longitudinally extending planar slit(s) formed through the wall(s) or body of a catheter tube having a closed distal end. The term “distal,” as used herein, refers to an end of the catheter that is inserted within the patient. In one embodiment, a distal end of a catheter including a valve structure may be positioned adjacent to a superior vena cava of a patient. When the valve(s) assume a “closed” position, the valve surfaces are opposed to seal against one another. The opposed surfaces of the slit(s) normally remain in sealing engagement, isolating the lumen(s) of the catheter from the region in the body of the patient outside the catheter tube.

Conventionally, the surfaces of a catheter valve may be coated with silicone oils. However, such silicone oils may be removed from the valve surfaces when the catheter is used for power injection. Explaining further, during power injection, flow rates may be between about 3 cubic centimeters per second to about 7 cubic centimeters per second or higher. It is believed that relatively high flow rates may remove at least a portion of conventionally provided silicone oils from the valve surfaces. Accordingly, such valves may not perform satisfactorily subsequent to (or during) power injection processes. For example, contrast media used in power injection can be “sticky” and may cause the valve surfaces to adhere to one another.

### SUMMARY

One aspect of the instant disclosure relates to a catheter comprising a body defining a lumen and a valve comprising a perforation through the body of the catheter, the perforation forming a plurality of catheter valve surfaces. Further, the catheter may comprise at least one coating substantially permanently adhered to at least a portion of at least one of the plurality of catheter valve surfaces. Such a configuration may provide a catheter valve with selected operational characteristics.

A further aspect of the instant disclosure relates to a method of at least partially coating a catheter valve surface. Particularly, a catheter may be provided, the catheter including a catheter valve comprising a perforation through the catheter, the perforation forming a plurality of catheter valve surfaces. Also, at least one coating may be substantially permanently adhered to at least a portion of at least one of the plurality of catheter valve surfaces.

Another aspect of the instant disclosure relates to an apparatus for at least partially opening a catheter valve. More specifically, the apparatus may comprise a tubular member, the tubular member including a bent region and an opening formed through the tubular member formed generally proximate the bent region. Further, the bent region and opening may be structured to cause a catheter valve of a catheter positioned within the tubular member to at least partially open.

Features from any of the above mentioned embodiments may be used in combination with one another in accordance with the instant disclosure. In addition, other features and advantages of the instant disclosure will become apparent to those of ordinary skill in the art through consideration of the ensuing description, the accompanying drawings, and the appended claims.

### BRIEF DESCRIPTION OF THE DRAWINGS

Advantages of the instant disclosure will become apparent upon review of the following detailed description and drawings, which illustrate representations (not necessarily drawn to scale) of various aspects of the instant disclosure, wherein:

FIG. 1 shows a partial side view of a catheter including a catheter valve;

FIG. 2 shows a cross-sectional view of the catheter shown in FIG. 1, taken through the catheter valve (along reference line A-A);

FIG. 3 shows a partial enlarged view of the catheter valve shown in FIG. 2 under closing forces;

FIG. 4 shows a cross-sectional view of the catheter shown in FIG. 1, wherein a pressure is developed within the lumen of the catheter;

FIG. 5 shows a partial enlarged view of the catheter valve shown in FIG. 2 under opening forces;

FIG. 6 shows a cross-sectional view of the catheter shown in FIG. 1, wherein fluid is flowing from the lumen of the catheter to the exterior of the catheter (e.g., infusion);

FIG. 7 shows a cross-sectional view of the catheter shown in FIG. 1, wherein a pressure is developed on an exterior of the catheter;

FIG. 8 shows a cross-sectional view of the catheter shown in FIG. 1, wherein fluid is flowing from an exterior of the catheter to the lumen of the catheter (e.g., aspiration);

FIG. 9 shows a partial side view of the catheter shown in FIG. 1, wherein the catheter valve is at least partially open and at least one of the catheter valve surfaces include a coating;

FIGS. 10-15 show various embodiments of catheter surfaces, wherein at least one of the catheter surfaces includes one or more coatings;

FIG. 16 shows another partial side view of the catheter shown in FIG. 1, wherein the catheter valve is at least partially open and the catheter is bent;

FIG. 17 shows one embodiment of an apparatus for at least partially opening a catheter valve of a catheter; and

FIG. 18 shows an assembly of a catheter including a catheter valve positioned within an apparatus as shown in FIG. 17.

### DETAILED DESCRIPTION

One aspect of the instant disclosure relates to catheters including a valve comprising a perforation through the catheter. More specifically, the instant disclosure contemplates that at least one coating may be formed upon at least a portion of one or more of a plurality of surfaces forming a catheter valve.

Catheters may be used for fluid communication with a vascular system of a patient. For example, a catheter may be useful for performing a therapeutic procedure occurring intermittently or more frequently, by way of example, in a superior vena cava of the venous subsystem of the cardiovascular system. In one embodiment, a catheter may include a distal portion that is intended to reside in a superior vena cava and a proximal end that resides outside the body of a patient. Of course, a proximal end of a catheter body may include a tubing clamp (e.g., a removable or nonremovable tubing clamp) and may terminate in a connector (e.g., luer or other known connection structure) that can be selectively coupled to medical equipment. In another embodiment, a proximal end of a catheter could be attached to a subcutaneously implantable access port, and the entire length of the catheter and the access port could be implanted within the body of a patient. In this configuration, the entire catheter may reside in the body.

Generally, a catheter, according to the instant disclosure, may comprise a relatively soft, biocompatible material and may define one or more lumens, without limitation. For example, such a catheter may comprise a medical grade silicone material. Silicone materials are relatively soft, relatively flexible for a wide range of temperatures, and may be free of harmful plasticizers. In addition, silicone materials may be generally resistant to chemicals, relatively nonthrombogenic, and atraumatic to surrounding tissues, all of which contribute to high biostability and biocompatibility. In addition, silicone materials may be sterilized by ethylene oxide gas, gamma or electron beam radiation, or steam autoclaving. In another embodiment, a catheter, according to the instant disclosure, may comprise a polyurethane material. Generally, polyurethane materials may exhibit relatively high tensile and tear strengths. Accordingly, a catheter comprising polyurethane may be more durable than a similarly sized catheter comprising silicone. A catheter constructed from a polyurethane material having a predetermined tensile strength may have a wall thickness that is less than the wall thickness of a catheter constructed from a silicone material having equal tensile strength. Fluid flow rates through a catheter lumen are proportional to the cross-sectional area thereof. The cross-sectional area of catheter lumens can be increased in catheters in which the outer wall thickness can be reduced. A cardiovascular access catheter constructed from a polyurethane material, therefore, can exhibit increased fluid flow rates relative to a similarly-sized silicone catheter.

A catheter should exhibit sufficient strength (e.g., wall thickness for a given material) to prevent tearing or bursting during use. For instance, catheters may be susceptible to bursting when fluids are injected through the catheter under pressure. The instant disclosure contemplates that a catheter may be structured to withstand pressures associated with flow rates up to approximately seven cubic centimeters per second or flow rates exceeding approximately seven cubic centimeters per second. In another embodiment, the catheter may be structured to withstand pressures associated with flow rates between approximately three cubic centimeters per second and approximately seven cubic centimeters per second. In addition, catheters may be susceptible to tearing during insertion into or removal from the body of the patient. For example, the portion of the catheter implanted in the body of the patient can tear at certain locations where the catheter is subjected to localized stress within the body. Further, the extracorporeal portion of an implanted catheter can tear due to mishandling.

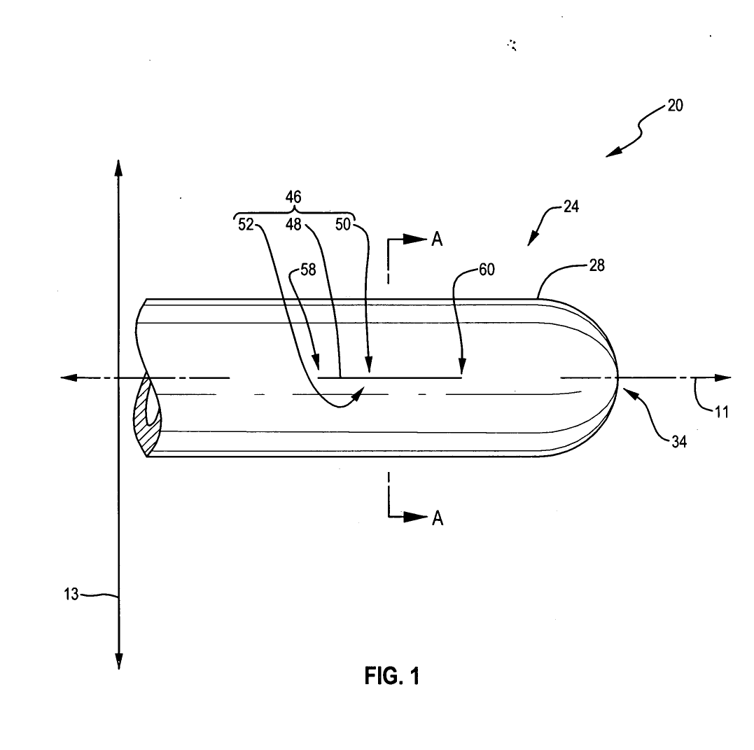

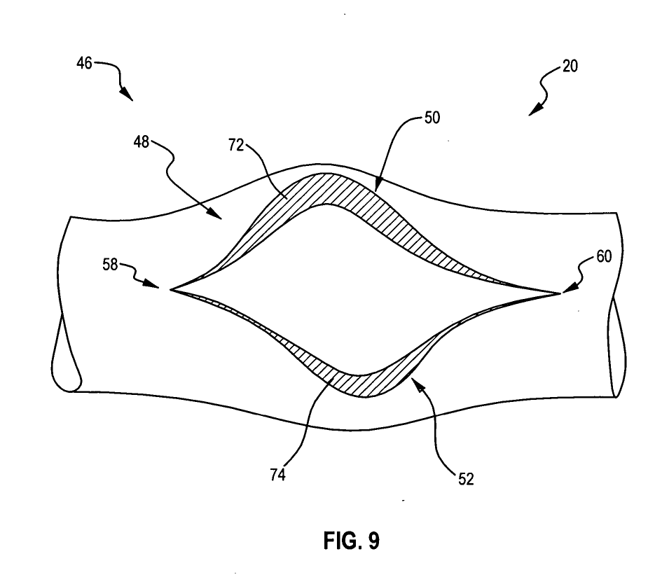

Generally, one aspect of the instant disclosure relates to coating at least a portion of at least one surface of a catheter valve comprising a perforation through a catheter with a selected material (e.g., a polymer, a metal, a ceramic). FIGS. 1-8 describe the structure and behavior of a catheter valve comprising a perforation through a catheter, with reference to the valve surfaces and operation of the valve. As described in greater detail in FIGS. 9-18, at least one surface of such a valve comprising a perforation through a catheter may be coated with at least one selected material.

More particularly, FIG. 1 shows an enlarged, partial view of a distal portion 24 of catheter 20. Catheter 20 is shown in FIG. 1 with reference to longitudinal axis 11 and lateral axis 13. As shown in FIG. 1, catheter 20 includes a closed distal tip 34. Catheter 20 may comprise any suitable material. For example, without limitation, catheter 20 may comprise silicone or polyurethane. In one embodiment, outer wall 28 of distal portion 24 of catheter 20 may be substantially cylindrical and distal tip 34 may be substantially semispherical. Further, catheter 20 includes a valve 46 comprising a perforation 48 formed through a wall or body of catheter 20 between a perforation end region 58 and a perforation end region 60. Perforation 48 may be formed at a selected position and along a selected path (e.g., a line, an arcuate path, a “zig-zag” path) through catheter 20. As shown in FIG. 1, valve 46 may be positioned proximate to distal tip 34. Explaining further, valve 46, in one embodiment, may comprise a substantially planar slit that extends longitudinally along outer wall 28 substantially parallel to longitudinal axis 11 of catheter 20. As may be understood, perforation 48, as shown in FIG. 1, forms a first valve surface 50 and a second valve surface 52.

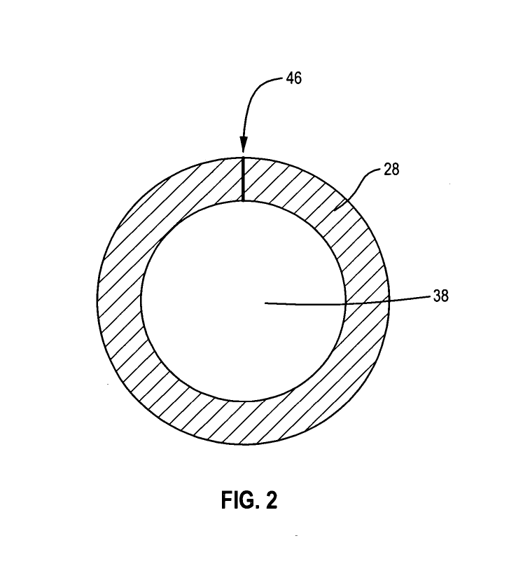

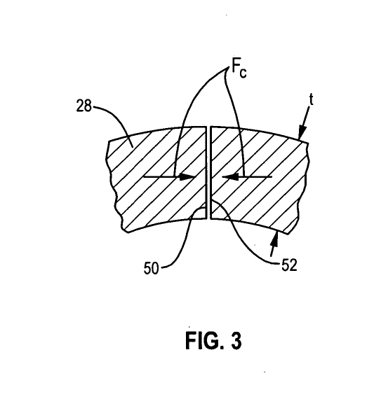

FIG. 2 shows a cross-sectional view of a portion of catheter 20 shown in FIG. 1 taken along section line A-A (FIG. 1). As shown in FIG. 2, outer wall 28 may enclose a single lumen 38. Of course, a catheter including multiple lumens may include a valve according to the instant disclosure, without limitation. FIG. 3 is an enlarged detail view of the portion of the cross-section shown in FIG. 2 depicting valve 46. As shown in FIGS. 2 and 3, valve 46 may include a first valve surface 50 and a second valve surface 52, which are adjacent to one another. Valve 46 may generally function as a two-way or bidirectional, normally-closed valve. In the closed position of valve 46, as shown in FIGS. 2 and 3, first valve surface 50 and second valve surface 52 of valve 46 are in sealing engagement. Thus, fluid may be precluded from entering or exiting lumen 38 of catheter device 20 through valve 46 in the closed position of valve 46, assuming that forces between first valve surface 50 and second valve surface 52 are sufficient to seal the first valve surface 50 and second valve surface 52 to one another. In addition, as shown in FIG. 3, closing forces F_{c} may be generated by the elastic “memory” of the material comprising the catheter 20. The closing forces F_{c} may facilitate the valve 46 closing or sealing. Accordingly, a radial thickness t of the catheter 20 and a length and configuration of the perforation 48 may influence the operational characteristics of the valve 46. Also, the instant disclosure recognizes that, cohesive, adhesive, combinations of adhesion and cohesion, absence of such attraction, or generally any factor affecting attraction between first valve surface 50 and second valve surface 52 may influence the operation of valve 46, as discussed in further detail below. Thus, one or both of a closing force F_{c} and an attractive force between the first valve surface 50 and second valve surface 52 may influence operational characteristics (e.g., opening behavior and closing behavior) of valve 46.

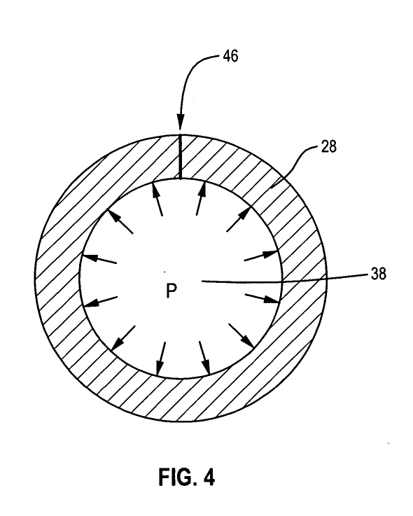

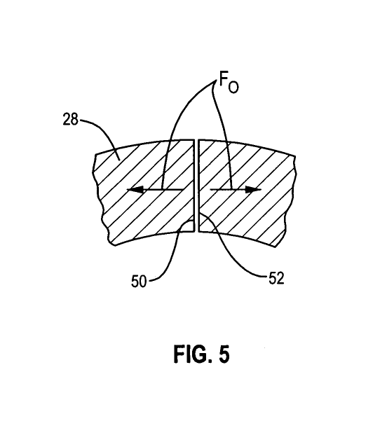

Valve 46 may be opened, in one example, by a positive pressure developed within lumen 38. Put another way, a positive pressure differential may be developed between lumen 38 (on one side of valve 46) and an exterior of catheter 20 (on another side of valve 46) (e.g., a region in the body of a patient). For example, FIG. 4 shows a positive pressure P developed within lumen 38 of catheter 20. As may be appreciated, force(s) (shown in one example by arrows in FIG. 4) may be developed on the interior surface(s) of catheter 20 due to pressure P. Such force(s) may cause the valve 46 to open. In further detail, FIG. 5 shows forces Fₒ, which may be developed by the positive pressure differential. It should be appreciated, however, that the stress state and forces developed near the valve 46 may be relatively complex. Therefore, the forces Fₒ are merely representations that may or may not completely describe the behavior of valve 46 when a positive pressure is developed within lumen 38.

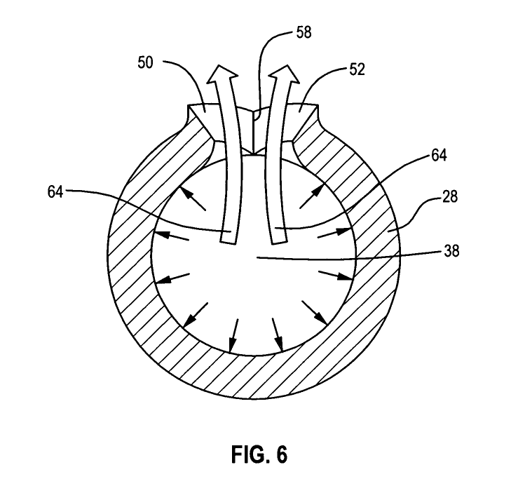

Explaining further, forces Fₒ may cause first valve surface 50 and second valve surface 52 to separate (e.g., by bending or other deformation of a wall of catheter 20), which compromises the sealing engagement of first valve surface 50 and second valve surface 52 shown in FIGS. 2 and 3. Thus, as shown in FIG. 6, first valve surface 50 and second valve surface 52 may be deformed or otherwise separated to open outwardly. Fluid 64 may be infused from lumen 38 to an exterior of the catheter 20 (e.g., into a cardiovascular system of a patient 10) due to a positive pressure differential. Of course, if the pressure differential between lumen 38 and the exterior of the catheter 20 is reduced to a threshold level, first valve surface 50 and second valve surface 52 may exhibit a closed position (e.g., a sealing engagement) as shown in FIGS. 2 and 3.

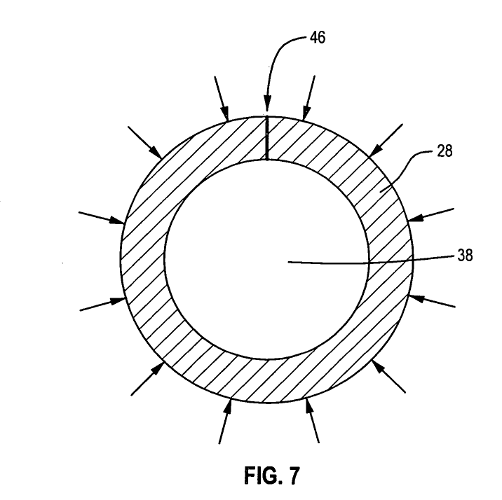

In another example, valve 46 may be opened by a negative pressure or suction developed within lumen 38, as shown in FIG. 7. Put another way, a positive pressure on the exterior of the catheter may open valve 46. Such a pressure environment may be described as a negative pressure differential between lumen 38 (on one side of valve 46) and an exterior of catheter 20 (on another side of valve 46) (e.g., a region in the body of a patient). As may be appreciated, force(s) (shown in one example by arrows in FIG. 7) may be developed on the interior surface(s) of catheter 20 due to pressure P. Such force(s) may cause the valve 46 to open. In further detail, FIG. 5 shows forces Fₒ, which may be developed by the negative pressure differential. It should be appreciated, however, that the stress state and forces developed near the valve 46 may be relatively complex. Therefore, the forces Fₒ are merely representations that may or may not completely describe the behavior of valve 46 when a negative pressure is developed within lumen 38.

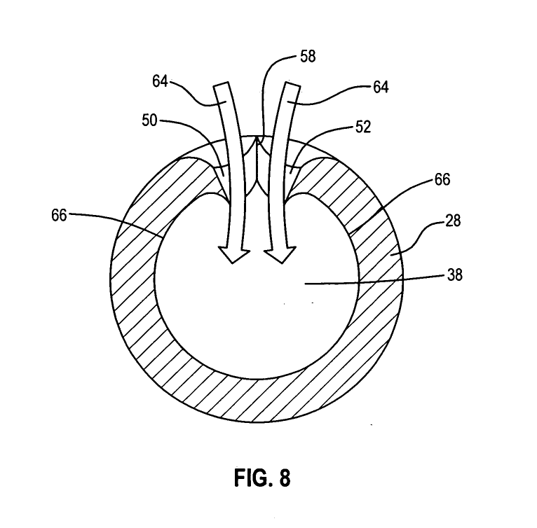

Explaining further, forces Fₒ may cause first valve surface 50 and second valve surface 52 to separate (e.g., by bending or other deformation of a wall of catheter 20), which allows for fluid to pass through valve 46 (e.g., by first valve surface 50 and second valve surface 52 shown in FIGS. 2 and 3). Thus, as shown in FIG. 8, first valve surface 50 and second valve surface 52 may be deformed or otherwise separated to open inwardly. Further, fluid 64 may be aspirated into lumen 38 from an exterior of the catheter 20 (e.g., from a cardiovascular system of a patient) due to the negative pressure differential. Of course, if the pressure differential between lumen 38 and the exterior of the catheter 20 is reduced to a threshold level, first valve surface 50 and second valve surface 52 may exhibit a closed position (e.g., a sealing engagement) as shown in FIGS. 2 and 3.

Thus, as may be appreciated by the above description, interaction between valve surfaces of a catheter valve may influence operation of the valve. In one example, a valve surface of a valve comprising a perforation through a silicone catheter may undesirably bond to one another (also termed “knitting,” as known in the art). The instant disclosure contemplates that at least a portion of a surface of a catheter valve comprising a perforation through a catheter may be coated with a material. In one embodiment, at least a portion of at least one surface of a catheter valve may be coated with a solid material. For example, at least a portion of a surface of a catheter valve may be coated with a polymer (e.g., a thermoplastic, a thermoset, or an elastomer). In one example, the instant disclosure contemplates that a portion of at least one surface of a catheter valve may be coated with polytetrafluoroethylene (PTFE), also known as TEFLON® fluoropolymer resin. For example, a coating of PTFE including a thickness of about 0.001 inches or less may be formed over at least a portion of a catheter valve surface. In another embodiment, a catheter valve may be coated with parylene. Parylene may be deposited by vacuum deposition, vapor deposition, or any other deposition process. Further, parylene deposition may form a substantially conformal coating or film over at least a portion of at least one valve surface of a catheter valve.

One aspect of the instant disclosure relates to adhering a coating or film to at least a portion of at least one catheter valve surface. More particularly, a material may be adhered to (i.e., have an affinity for, etc.) at least a portion of at least one catheter valve surface by attractive forces (e.g., surface tension/energy, adhesion, cohesion, intermolecular forces) having a magnitude (cumulative, if more than one) that exceeds a nominal magnitude of attractive forces (e.g., affinity) developed between a silicone oil and the at least a portion of the at least one catheter valve. In one embodiment, a coating or film may exhibit an affinity for at least a portion of a catheter valve surface that exceeds a naturally occurring affinity of a fluid (e.g., a silicone oil) for the at least a portion of a catheter valve surface. In another embodiment, a coating or film may be described as substantially affected (e.g., removed, diminished, or damaged) by flow of a fluid through the catheter valve. Put another way, such a coating or film may be worn (e.g., contact between the coating and a material having a hardness equal to or exceeding a hardness of the coating) or eroded (e.g., solids flowing within a fluid passing through a catheter valve), but may not be substantially removed from the catheter valve surface, diminished, or damaged due solely to a fluid flowing through the valve. Thus, in one embodiment, adherence of the coating as well as the structural properties (e.g., strength, modulus of elasticity) of the coating may resist removal or other damage in response to operation of the catheter valve.

Explaining further, catheter 20 is shown in a partial, enlarged view in FIG. 9. As shown in FIG. 9, first valve surface 50 and second valve surface 52 meet or originate at a perforation end region 58 and at a perforation end region 60. Thus, the inward or outward deflection or deformation (in relation to the closed state of the valve 46) of the valve 46 may vary along the length of perforation 48. As shown in FIG. 9, a maximum deflection or deformation may occur generally near the center of the length of perforation 48 and may decrease with distance away from the center of the perforation 48 in the direction of each of perforation end region 58 and 60, respectively. Generally, at least a portion of first valve surface 50, second valve surface 52, or both may be coated with a selected material. In one embodiment, as shown in FIG. 9, first valve surface 50 may be substantially covered with a coating 72 and second valve surface 52 may be substantially covered with a coating 74. In one embodiment, coating 72 and coating 74 may comprise PTFE. Such a configuration may provide a valve 46 that is able to function satisfactorily subsequent to power injection (e.g., flow rates of at least about 3 cubic centimeters per second or greater) and may perform a plurality of power injection processes satisfactorily. In one embodiment, coatings 72 and 74 may be formulated to inhibit undesirable attraction (e.g., adhesion, cohesion, knitting) between the first valve surface 50 and second valve surface 52. More generally, coatings 72 and 74 may be formulated to produce a selected level of attraction forces (e.g., by increasing or decreasing such forces) between first valve surface 50 and second valve surface 52. Further, coatings 72 and 74 may be relatively well-adhered to first valve surface 50 and second valve surface 52, respectively. Also, optionally, coatings 72 and 74 may be formulated to protect first valve surface 50 and second valve surface 52.

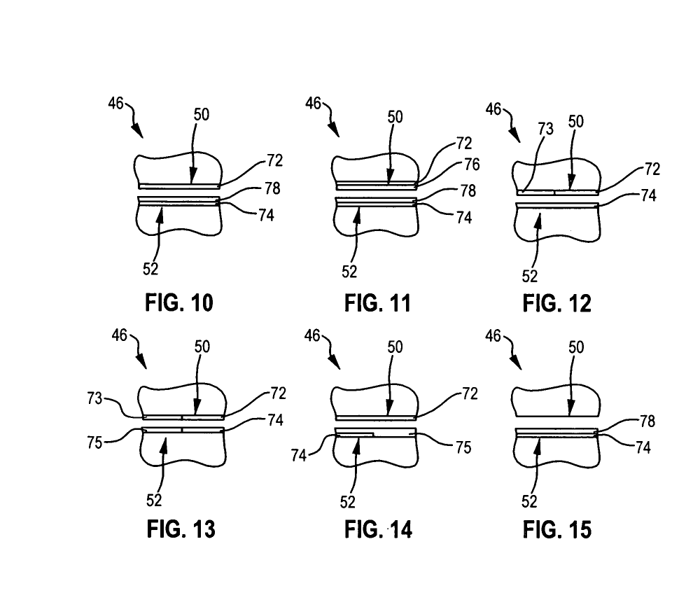

Many different configurations and variations for coating at least a portion of one or more surfaces of a catheter valve are encompassed by the instant disclosure. For instance, a plurality of coatings may be applied to one or more surfaces comprising a catheter valve. More specifically, FIGS. 10-15 illustrate some examples of one or more catheter valve surfaces with various coating configurations. For example, FIG. 10 shows a schematic, side cross-sectional view of a catheter valve 46 including catheter valve surfaces 50 and 52. As shown in FIG. 10, catheter valve surface 50 includes coating 72 and catheter valve surface includes coatings 74 and 78. In one embodiment, each of coatings 72, 74, and 78 may comprise a different material, without limitation. FIG. 11 shows a schematic side cross-sectional view of a catheter valve 46 including catheter valve surface 50 and 52, wherein catheter valve surface 50 includes coatings 72 and 76 and catheter surface 52 includes coatings 74 and 78. Thus, in one embodiment, opposing catheter valve surfaces 50 and 52 may each include a plurality of coatings, if desired. Further, in one embodiment, each of coatings 72, 74, 76, and 78 may comprise different materials, without limitation. In another embodiment, coatings 72 and 74 may comprise one material and coatings 76 and 78 may comprise a second material. FIG. 12 shows a schematic side cross-sectional view of a further embodiment of a catheter valve 46. Particularly, as shown in FIG. 12, catheter surface 50 includes coatings 72 and 73, while catheter surface 52 includes coating 74. As shown in FIG. 12, it should be appreciated that coatings 72 and 73 may be positioned generally adjacent to one another and may, optionally, comprise different materials, without limitation. For example, coatings 72 and 73 may abut one another or may be separated from one another upon surface 50, without limitation. FIG. 13 shows another embodiment of a catheter valve 46 including catheter valve surfaces 50 and 52, wherein catheter valve surface 50 includes coatings 72 and 73, which are positioned adjacent to one another and catheter valve surface 52 includes coatings 74 and 75, which are positioned adjacent to one another. In one embodiment, coatings 72, 73, 74, and 75 may comprise different materials, without limitation. In another embodiment, one coating may be formed over at least a portion of another coating and at least a portion of a catheter valve surface. For example, FIG. 14 shows a schematic side cross-sectional view of a catheter valve 46 including catheter valve surface 50 and 52. As shown in FIG. 14, catheter valve surface 50 includes coating 72 and catheter valve surface 52 includes coatings 74 and 75, wherein coating 75 is formed over at least a portion of coating 74 and a portion of catheter valve surface 52. Of course, coatings 72, 74, and 75 may comprise different materials. As mentioned above, generally, at least one catheter valve surface may be coated. More particularly, FIG. 15 shows a schematic, side cross-sectional view of a catheter valve 46 including catheter valve surface 50 and catheter valve surface 52, wherein catheter valve surface 52 includes coatings 74 and 78.

The instant disclosure further contemplates a method for coating at least a portion of a catheter valve surface. Generally, at least one coating may be formed over at least a portion of at least one of a plurality of catheter valve surfaces. Particularly, the plurality of catheter valve surfaces may be separated and at least one catheter valve surface may be at least partially coated. In one embodiment, a catheter valve comprising a perforation through a catheter may be at least partially opened and at least one coating may be formed upon at least a portion of a surface comprising the catheter valve. At least one coating at least partially covering a valve surface of a catheter valve may be formed by any process as known in the art. For example, spraying, dipping, sputtering, chemical vapor deposition, electroless deposition, chemical treating, or any other deposition process may be used for forming a coating may be utilized for forming at least one coating over at least a portion of a catheter valve surface. Further, it should be understood that masking technologies such as those employed for forming layers (metal or otherwise) (e.g., for semiconductor manufacturing) may be employed for forming one or more coating layers upon at least a portion of a catheter valve surface.

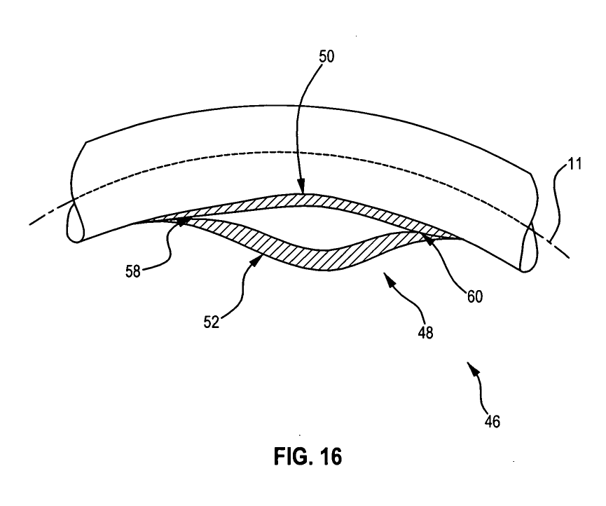

Generally, various actions may at least partially separate catheter valve surfaces comprising a catheter valve. For example, a portion of a catheter may be deformed (e.g., pinched, flattened) to cause catheter valve 46 to at least partially open (e.g., inwardly or outwardly). In one example, the instant disclosure contemplates that a catheter valve may be at least partially opened by bending a region of the catheter. In one embodiment, a region including the catheter valve may be bent to cause the catheter valve to at least partially open. FIG. 16 shows a partial, enlarged perspective view of a catheter 20 that is bent (e.g., longitudinal axis 11 is arcuate). Generally, catheter 20 may be bent so that end regions 58 and 60 are caused to move closer to one another in comparison to a distance between end regions 58 and 60 when the catheter valve 46 is closed. Thus, as catheter valve 46 is at least partially opened, catheter valve surfaces 50 and 52 may become more accessible or exposed. Accordingly, at least one coating may be formed over at least a portion of one or both of catheter surfaces 50 and 52.

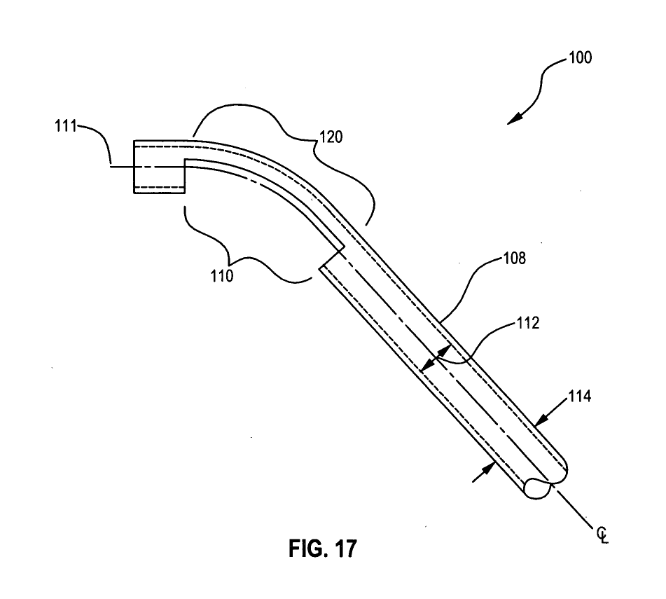

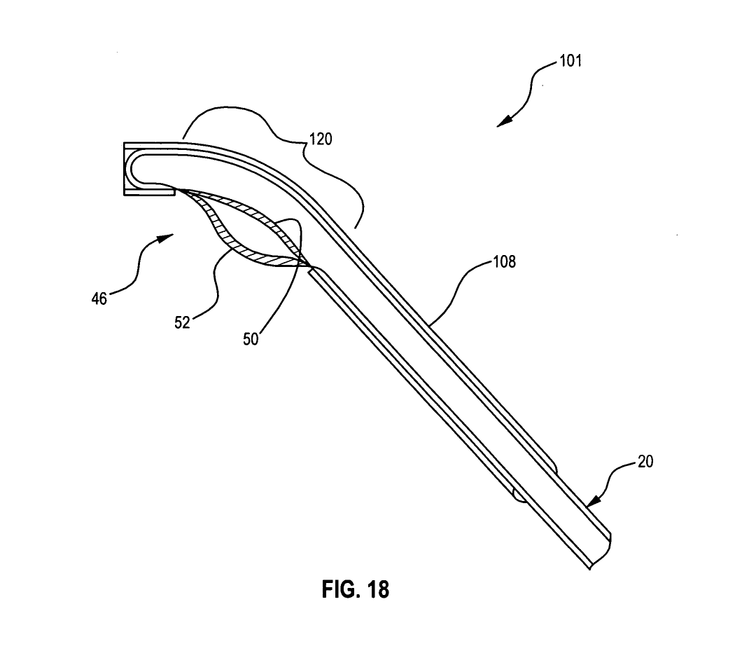

Further, an apparatus for holding or securing a catheter including a catheter valve to cause the catheter valve to at least partially open is contemplated by the instant disclosure. More particularly, an apparatus may be configured for at least partially deforming a catheter to at least partially open a catheter valve. FIG. 17 shows a schematic view of an apparatus 100 configured for bending at least a portion of a catheter to at least partially open a catheter valve formed in the catheter. As shown in FIG. 17, apparatus 100 may comprise a tubular member 108 defining an inner dimension 112 (e.g., a diameter) and an outer dimension (e.g., a diameter) 114. Further, tubular member 108 may include bent region 120. Bent region 120 may be structured to cause a catheter valve of a catheter positioned within tubular member 108 to at least partially open. In addition, an opening 110 may be formed through a portion of the tubular member 108. Opening 110 may be configured for allowing access to a catheter valve of a catheter positioned generally within tubular member 108. In further detail, FIG. 18 shows a schematic view of an assembly 101 including a catheter 20 positioned within apparatus 100. As shown in FIG. 18, positioning catheter 20 appropriately within tubular member 108 may cause the catheter 20 to bend and at least partially open catheter valve 46. Thus, catheter valve surfaces 50 and 52 may be at least partially accessible or exposed. Such a configuration may facilitate forming at least one coating over at least a portion of one or both of catheter valve surfaces 50 and 52. It may be desirable to limit an amount of time that a catheter is positioned within tubular member 108, so that permanent deformation may be minimized or avoided.

Any of the catheters disclosed herein may include a plurality of lumens or a single lumen. One or more catheter valve(s) may be formed through a catheter wall in communication with one or more of a plurality of lumens comprising a catheter. Thus, in one example, a plurality of catheter valves may be formed for fluid communication with a selected lumen of a catheter (e.g., a single lumen catheter or a multiple lumen catheter). Further, in another example, a plurality of catheter valves may be formed for fluid communication with each of a plurality of selected lumens of a catheter. In yet an additional example, one catheter valve may be provided for each of a plurality of lumens within a catheter. Other combinations or variations of a catheter including at least one lumen and at least one catheter valve may be apparent to one of ordinary skill in the art. Further, the instant disclosure encompasses any catheter including one or more lumens and one or more catheter valves, wherein at least a portion of at least one catheter valve surface includes a coating, without limitation.

While certain representative embodiments and details have been shown for purposes of illustrating aspects of the instant disclosure, it will be apparent to those skilled in the art that various changes in the methods and apparatus disclosed herein may be made without departing form the scope of the instant disclosure, which is defined in the appended claims. For example, other catheter materials, lumen configurations, and shapes may be employed. Further, various other materials and methods may be employed for forming at least one coating over at least one catheter valve surface of a catheter valve of the instant disclosure. The words “including” and “having,” as used herein including the claims, shall have the same meaning as the word “comprising.”

What is claimed is:

1. A catheter comprising:
a body defining a lumen;
at least one valve comprising at least one perforation through the body of the catheter, the at least one perforation forming a plurality of catheter valve surfaces; and
at least one coating substantially permanently adhered to at least a portion of at least one of the plurality of catheter valve surfaces.

2. The catheter of claim 1, wherein the at least one coating comprises a polymer.

3. The catheter of claim 1, wherein the at least one coating comprises at least one of the following: thermoplastic, a thermoset, and an elastomer.

4. The catheter of claim 1, wherein at least a portion of the body is configured to accommodate fluid flow rates up to approximately seven cubic centimeters per second.

5. The catheter of claim 1, wherein the perforation is substantially planar and substantially parallel to a longitudinal axis of the catheter.

6. The catheter of claim 1, wherein the plurality of catheter valve surfaces comprise two catheter valve surfaces, each of which are substantially planar.

7. The catheter of claim 1, wherein the at least one coating comprises a plurality of coatings.

8. The catheter of claim 7, wherein at least two of the plurality of coatings are adjacent to one another.

9. The catheter of claim 7, wherein at least one of the plurality of coatings is formed over at least a portion of another of the plurality of coatings.

10. The catheter of claim 7, wherein each of the plurality of coatings comprises a different material.

11. The catheter of claim 1, wherein the at least one coating adheres to the at least a portion of the at least one of the plurality of catheter valve surfaces by exceeding the naturally occurring affinity of a fluid for the at least a portion of the at least one of the plurality of catheter valve surfaces.

12. The catheter of claim 1, the body defines a plurality of lumens.

13. The catheter of claim 12, wherein the catheter further comprises a plurality of valves, each of the plurality of valves comprising at least one perforation formed through the body of the catheter and in fluid communication with at least one of the plurality of lumens.

14. The catheter of claim 13, wherein at least one coating is substantially permanently adhered to the plurality of valves.

15. A method comprising:
providing a catheter including at least one catheter valve comprising at least one perforation through the catheter, the at least one perforation forming a plurality of catheter valve surfaces; and
substantially permanently adhering at least one coating to at least a portion of at least one of the plurality of catheter valve surfaces.

16. The method of claim 15, further comprising separating the plurality of catheter surfaces, wherein separating the plurality of catheter valve surfaces comprises at least partially opening the catheter valve.

17. The method of claim 15, wherein separating the plurality of catheter valve surfaces comprises deforming the catheter to cause the at least one catheter valve to at least partially open.

18. The method of claim 16, wherein deforming the catheter to cause the at least one catheter valve to at least partially open comprises bending the catheter to cause the at least one catheter valve to at least partially open.

19. The method of claim 15, wherein substantially permanently adhering at least one or more coating comprises substantially permanently adhering at least one or more coatings comprising at least one of the following: a thermoplastic, a thermoset, and an elastomer.

20. The method of claim 15, further comprising:
placing a distal end of the catheter into a vasculature of a patient;
connecting a proximal end of the catheter to a fluid source; and
injecting fluid from the fluid source into the patient via the catheter at a flow rate up to approximately seven cubic centimeters per second.

21. An apparatus for at least partially opening a catheter valve, the apparatus comprising:
a tubular member, the tubular member including a bent region;
an opening formed through the tubular member formed generally proximate the bent region;
wherein the bent region and opening are structured to cause a catheter valve of a catheter positioned within the tubular member to at least partially open.
